(19) United States
(12) Patent Application Publication
(10) Pub. No.: US 2012/0296216 A1
(43) Pub. Date: Nov. 22, 2012

(54) METHODS AND SYSTEMS OF AIMING SENSOR(S) FOR MEASURING CARDIAC PARAMETERS

(75) Inventors: Yehuda SHARF, Tel-Aviv (IL); Shimon Hayun, Rosh HaAyin (IL); Eldad Tzadok, Herzlia (IL); Yotam Luz, Moshav Burgata-Doar-Na Lev HaSharon (IL)

(73) Assignee: Cardiogal Ltd., Tel-Aviv (IL)

(21) Appl. No.: 13/108,030

(22) Filed: May 16, 2011

(51) Int. Cl. A61B 8/06; A61B 8/00

(52) U.S. Cl. 600/454; 600/459; 600/453

(57) ABSTRACT

A placement mechanism for placing a sensor for non invasive measurement of at least one parameter. The placement mechanism comprises a angling unit which angles a sensor in relation to a sensor positioning site on a skin of a target patient in proximity to the ribs while maintaining a front side contact zone of the sensor in contact with the sensor positioning site and an attachment element for attaching the placement mechanism to a body of the target patient so that the front side contact zone being in contact with the sensor positioning site.

### FIELD AND BACKGROUND OF THE INVENTION

The present invention, in some embodiments thereof, relates to a method and a system for aiming biological sensors and, more particularly, but not exclusively, to a methods and systems for aiming sensor(s) for measuring cardiac parameters.

Patients recovering from cardiac surgery and those admitted to Intensive Care Units (ICU's) with heart failure often require hemodynamic (blood flow and pressure) monitoring which usually involves a Swan-Ganz catheter that requires an invasive procedure for placement. During the last years, various devices and procedures for facilitating non-invasive cardiac monitoring and measurement have been developed. By using such devices infection risks typically associated with Swan-Ganz catheterization are avoided.

At present, the preferred non-invasive alternative method for making these measurements is a high-end echocardiograph machine specially equipped with tissue Doppler imaging. Echocardiography is the gold standard imaging technique for detecting mechanical heart disease in the clinical setting. It is used to measure ejection fraction, dimensions, thickness and movement of the ventricles. The “Doppler Effect” has been used in echocardiography for many years, in which frequency shifts of ultrasound waves are used to calculate blood flow velocities and direction in the heart chambers. Tissue Doppler echocardiography (TDE) is a relatively recent addition to the diagnostic ultrasound examination; it permits an assessment of heart muscle motion using Doppler shifts. Heart muscle motion is characterized by low velocity (of the order of 0.1 meter/second) relative to the velocity of blood flow (typically of the order of 1 meter/second), and by much higher intensity of echo amplitude (+40 dB). “Recommendations for Quantification of Doppler Echocardiography: A Report From the Doppler Quantification Task Force of the Nomenclature and Standards Committee of the American Society of Echocardiography”, by Miguel A. Quiñones, Catherine M. Otto, Marcus Stoddard, Alan Waggoner, and William A. Zoghbi, (J Am Soc Echocardiogr 2002; 15:167-84), the disclosure of which is incorporated herein by reference provides various definitions for Doppler Echocardiography, which may be useful in the practice of some embodiments of the invention.

Doppler techniques measure the motion of blood and muscle tissues. The Doppler technique allows the quantitative assessment of heart muscle contraction and relaxation velocities. In addition, the Doppler technique has greater temporal resolution (ability to detect rapidly occurring events) than standard 2-dimensional echocardiography. This feature allows for more accurate “timing” of various events in normal (and abnormal) heart contraction and relaxation.

Standard transmitral flow velocity profiles have been shown to be very dependent on loading pressures; “preload” is the pressure pushing the blood through the mitral valve (i.e. left atrial pressure). This effect of preload is particularly important in the E wave velocity (Doppler flow wave corresponding to early diastole filling of the left ventricle (LV) in diastole). The higher the preload (i.e. left atrial diastole pressure), the higher the E wave velocity. It has also been shown that E′ (tissue Doppler wave corresponding to early diastole relaxation of the LV) is relatively load independent and that the ratio of E/E′ is well correlated with the left ventricular diastole pressures (LVDP). S′ (tissue Doppler wave corresponding to LV systolic contraction) measures the strength of myocardial contraction and can be used to assess myocardial function during systole. See also “Recommendations for the Evaluation of Left Ventricular Diastolic Function by Echocardiography”, Guidelines and Standards of American Society of Echocardiography, by Sherif F. Nagueh, Christopher P. Appleton, Thierry C. Gillebert, Paolo N. Marino, Jae K. Oh, Otto A. Smiseth, Alan D. Waggoner, Frank A. Flachskampf, Patricia A. Pellikka, and Arturo Evangelista, which the disclosure of which is incorporated herein by reference and provides definitions and recommendations on utility for various Doppler indices, which may be useful in the practice of some embodiments of the invention.

### SUMMARY OF THE INVENTION

According to some embodiments of the present invention there is provided a placement mechanism for placing a sensor for non invasive measurement of at least one parameter, comprising:

a angling unit which angles a sensor in relation to a sensor positioning site on a skin of a target patient in proximity to the ribs while maintaining a front side contact zone of the sensor in contact with the sensor positioning site; and

an attachment element for attaching the placement mechanism to a body of the target patient so that the front side contact zone being in contact with the sensor positioning site.

Optionally, the placement mechanism further comprises an elevation element for adjusting the distance between the front side contact zone and the sensor positioning site.

Optionally, the placement mechanism further comprises a label having a first fastening element on a front side and a coating of adhesive on a back side, the label being sized and shaped to be temporarily attached to the skin, at least partly around the sensor positioning site; the attachment element having a second fastening element configured for being detachably connected to the first fastening element.

More optionally, the label has an opening to allow placing the front side contact zone in contact with the sensor positioning site.

More optionally, the angling unit comprises a

housing for supporting the sensor and a substantially hemispherical element forming a joint with the housing so as to allow angling the hemispherical housing with the sensor; wherein the second fastening element being connected to the hemispherical element.

More optionally, the angling unit comprises a shaft connected to the housing; wherein the substantially hemispherical element has a slit for facilitating the angling of the shaft.

Optionally, the placement mechanism further comprises at least one locking element for fixating the sensor in a signal capture angle while maintaining the front side contact zone in contact with the sensor positioning site.

Optionally, the angling unit comprises a slidable element configured to be maneuvered in parallel to the a sliding plane so as to adjust a signal capture angle of the sensor in relation to the sliding plane while maintaining the front side contact zone in contact with the sensor positioning site.

Optionally, the angling unit comprises actuation means for angling the sensor according to instructions from a monitoring device analyzing an output of the sensor.

According to some embodiments of the present invention there is provided a placement mechanism for placing a sensor for non invasive measurement of at least one parameter. The placement mechanism comprises a angling unit which simultaneously angles a plurality of sensors in relation to a plurality of sensor positioning sites on a skin of a target patient, while maintaining a front side contact zone of each sensor in contact with a respective the sensor positioning site and an attachment element for attaching the placement mechanism to a body of the target patient so that the front side contact zone being in contact with the sensor positioning site.

More optionally, the angling unit comprises a plurality of stirring arms, each stirring arm being mechanically connected, at a first end, to a stir junction and to one of the plurality of sensors at a second end.

More optionally, each second end is mechanically connected to a respective the sensor via a rotating joint that rotates around an axis perpendicular to a respective the stirring arm.

More optionally, at least one of the plurality of stirring arms is telescopic.

According to some embodiments of the present invention there is provided a method for placing at least one sensor for non invasive measurement of at least one parameter. The method comprises providing at least one ultrasound transducer having a front side contact zone configured for being in contact with at least one sensor positioning site on the skin of a target patient, attaching the at least one ultrasound transducer to a body of the target patient, identifying signal based location indication by analyzing an ultrasound signal received from the ultrasound transducer, adjusting, according to the signal based location indication, a signal capture angle of the at least one sensor in relation to the skin while maintaining the front side contact zone in contact with the target area, and fixating the at least one sensor in the signal capture angle, while maintaining the front side contact zone in contact with the target area.

Optionally, the identifying comprises extracting a blood motion and a tissue motion from the ultrasound signal and performing the identifying by identifying a correlation between the blood motion and the tissue motion.

Optionally, the signal based location indication is a trace indicative of a mitral valve opening which is extracted from the ultrasound signal.

More optionally, the method further comprises monitoring a plurality of cardiac parameters according to an analysis of the ultrasound signal.

More optionally, the method further comprises identifying the at least one sensor positioning site according to a time velocity Doppler trace of an echocardiography imaging probe.

Optionally, the adjusting comprises presenting a plurality of indications each indicative of the detection of an interim signal based locator in the ultrasound signal.

More optionally, the method further comprises acquiring an electrocardiogram (ECG) signal; wherein the adjusting is performed according to the ECG signal.

Optionally, the identifying comprises identifying the following: a) systolic and diastoles in the ultrasound signal, b) an indication of a mitral valve opening in the ultrasound Doppler signal during the diastole, c) a plurality of onsets of a blood inflow in the ultrasound Doppler signal during the diastole, and d) a repetitive pattern of the plurality of onsets during at least one respiration cycle of the target patient. Each member of the a-d may not be identified if a proceeding member of the a-d has been identified.

According to some embodiments of the present invention there is provided a method for aiming at least one ultrasound transducer for measuring at least one parameter. The method comprises attaching at least one ultrasound transducer to a body of a target patient in front of at least one sensor positioning site, receiving an ultrasound Doppler signal from the at least one ultrasound transducer, tuning a transmission/reception angle of the at least one ultrasound transducer until the following are being identified: a) systolic and diastoles in the ultrasound Doppler signal, b) an indication of at least one mitral valve opening in the ultrasound Doppler signal during the diastole, c) a plurality of onsets of a blood inflow in the ultrasound Doppler signal during the systole, d) a repetitive pattern of the plurality of onsets during at least one respiration cycle of the target patient, and fixating the at least one ultrasound transducer in the tuned transmission/reception angle in front of the at least one sensor positioning site. Each member of the a-d may not be identified if a proceeding member of the a-d has been identified.

Optionally, the tuning is performed while maintaining a front side contact zone of each ultrasound transducer in contact with a respective the transducer positioning site.

According to some embodiments of the present invention there is provided a method for

monitoring a patient. The method comprises fixating each of a plurality of ultrasound transducers in a signal capture angle so that a front side contact zone thereof is in contact with one of a plurality of sensor positioning sites on the skin of a target patient and non invasively measuring, using the plurality of ultrasound transducers, at least one cardiac parameter in a plurality of sessions. Each one of the plurality of ultrasound transducers remains fixated in a respective the signal capture angle during the monitoring period.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or an operator input device such as a keyboard or mouse are optionally provided as well.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method of aiming sensors, such as one or more ultrasound probes, for non invasive measurement of one or more cardiac parameters, according to some embodiments of the present invention;

FIG. 2 is an exemplary monitoring system including a monitor device and a placement mechanism, according to some embodiments of the present invention;

FIG. 3 is a schematic illustration of a thorax and exemplary sensor positioning sites which are marked thereon (dots), according to some embodiments of the present invention;

FIG. 4 is a table defining a predefined order, for example preferred and/or suggested order, for placing sensors in potential sensor positioning sites, according to some embodiments of the present invention;

FIGS. 5A-5C, are schematic illustration of components of a placement mechanism 500, for example as depicted in FIG. 5D, according to some embodiments of the present invention;

FIG. 5D is a schematic illustration of an exemplary placement mechanism, according to some embodiments of the present invention;

FIG. 6A is a schematic illustration of a placement mechanism that allows adjusting the signal capture angle of a sensor for non invasive measurement of cardiac parameter(s), according to some embodiments of the present invention;

FIGS. 6B-6C are schematic illustration of the placement mechanism depicted in FIG. 6A, aiming the front side of the sensor to different directions, according to some embodiments of the present invention;

FIG. 7A depicts a schematic illustration of a placement mechanism, according to some embodiments of the present invention;

FIG. 7B is a sectional schematic illustration of the placement mechanism depicted at FIG. 7A, according to some embodiments of the present invention;

FIG. 7C is a sectional schematic illustration of a angling shaft, according to some embodiments of the present invention;

FIG. 8 is a schematic illustration of a stirring mechanism that is connected to three sensors and allows an operator to tilt them simultaneously by stirring a handle located at a contact point, according to some embodiments of the present invention;

FIG. 9 is a schematic illustration of a blowup of a connection of arms in the stirring mechanism depicted in FIG. 8, according to some embodiments of the present invention;

FIGS. 10A and 10B depicts a plurality of Doppler ultrasound signals, gated according to systole and diastole, whereas transition from diastole to systole corresponds to transition from negative to positive tissue Doppler velocities, according to some embodiments of the present invention;

FIG. 10C is an ECG signal, captured with the plurality of Doppler ultrasound signals, which is used to mark the start of systole by a QRS detection, according to some embodiments of the present invention;

FIG. 11 is a flowchart of a method for detecting a signal based location indication which is indicative of a potential signal capture angle for a sensor, according to some embodiments of the present invention;

FIGS. 12A and 12B depicts a plurality of Doppler ultrasound signals, gated according to systole and diastole, wherein a signal segment indicative of a mitral valve opening is emphasized, according to some embodiments of the present invention;

FIG. 12C is an ECG signal captured with the plurality of Doppler ultrasound signals depicted in FIGS. 12A and 12B, according to some embodiments of the present invention;

FIGS. 12D and 12E are schematic illustrations of traces which are extracted from one or more received Doppler echocardiography signals and an ECG signal or a processing thereof, according to some embodiments of the present invention;

FIG. 13 is a monochromatic M-Mode view of emphasized blood inflow;

FIG. 14 is an image of an enhanced Doppler trace of blood and tissue that is displayed for any chosen range gate and generated using a non linear (logarithmic) color scheme to show concurrent events of relatively strong tissue signals and relatively weak blood signals, according to some embodiments of the present invention; and

FIG. 15 is an image of a segment of an enhanced Doppler trace of blood and tissue that depicts the distance between the onset of E and the onset of E′, according to some embodiments of the present invention.

### DESCRIPTION OF EMBODIMENTS OF THE INVENTION

The present invention, in some embodiments thereof, relates to a method and a system for aiming biological sensors and, more particularly, but not exclusively, to a methods and systems for aiming sensor(s) for measuring cardiac parameters.

According to some embodiments of the present invention there is provided a placement mechanism that places sensor(s) for non invasive measurement of one or more cardiac parameters, such as ultrasound transducers, on the chest of a patient. The placement mechanism may be provided as a part of a monitoring system that analyzes the outputs of the sensors or as an addition (add-on) to existing monitoring systems. The placement mechanism comprises a angling unit which angles a sensor, such as an ultrasound transducer, in relation to a sensor positioning site on a skin of a target patient while it maintains a front side contact zone thereof in contact with the sensor positioning site and an attachment element that attaches the placement mechanism to a body of the target patient so that the front side contact zone is in contact with the sensor positioning site. The angling unit optionally includes a sticker having a first fastening element on a front side and a coating of adhesive on a back side. The sticker is designed to be temporarily attached to the skin of the patient, at least partly around the sensor positioning site, for example therearound. In such an embodiment, the attachment element includes a second fastening element for detachably connect the placement mechanism to the first fastening element so that the front side contact zone of the sensor is in contact with the sensor positioning site. The angling unit optionally includes a slidable element, such as a plate, that slide along a sliding plane and tilt a shaft that is connected to the sensor so that the front side contact zone of the sensor is in contact with the sensor positioning site.

According to some embodiments of the present invention, the placement mechanism includes a angling unit that simultaneously angles a plurality of sensors in relation to a plurality of sensor positioning sites on a skin of a target patient while maintaining a front side contact zone of each one of them in contact with one of the sensor positioning sites. The angling unit optionally comprises a plurality of stirring arms which are connected at a common stirring junction. Each one of the stirring arms is optionally connected to a rotary joint, optionally with two rotation axes, which facilitates simultaneously adjusting the signal capture angles of the sensors while maintaining a front side contact zone of each one of them in contact with the sensor positioning sites.

According to some embodiments of the present invention, there is provided a method of aiming an ultrasound transducer for measuring one or more cardiac parameters. The method is based on attaching ultrasound transducer(s) to a body of a target patient in front of and optionally in a direct contact with sensor positioning site(s) and receiving an ultrasound Doppler signal from said the ultrasound transducer(s). The signal capture angle of each one of the sensors is tuned according to signal based location indications which are identified in the ultrasound Doppler signal. The signal based location indications are gated systolic and diastole periods, one or more indications of a mitral valve opening during the diastole, an onset of a blood inflow during the systole, and a repetitive pattern of these onsets during more than heartbeats, or one or more respiration cycles of the target patient. Now, the tuned signal capture angle of the ultrasound transducer(s) is fixated in front of the at least one sensor positioning site, optionally in contact therewith. The angling is optionally performed while maintaining a front side contact zone of each one of the sensors in contact with the sensor positioning sites.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

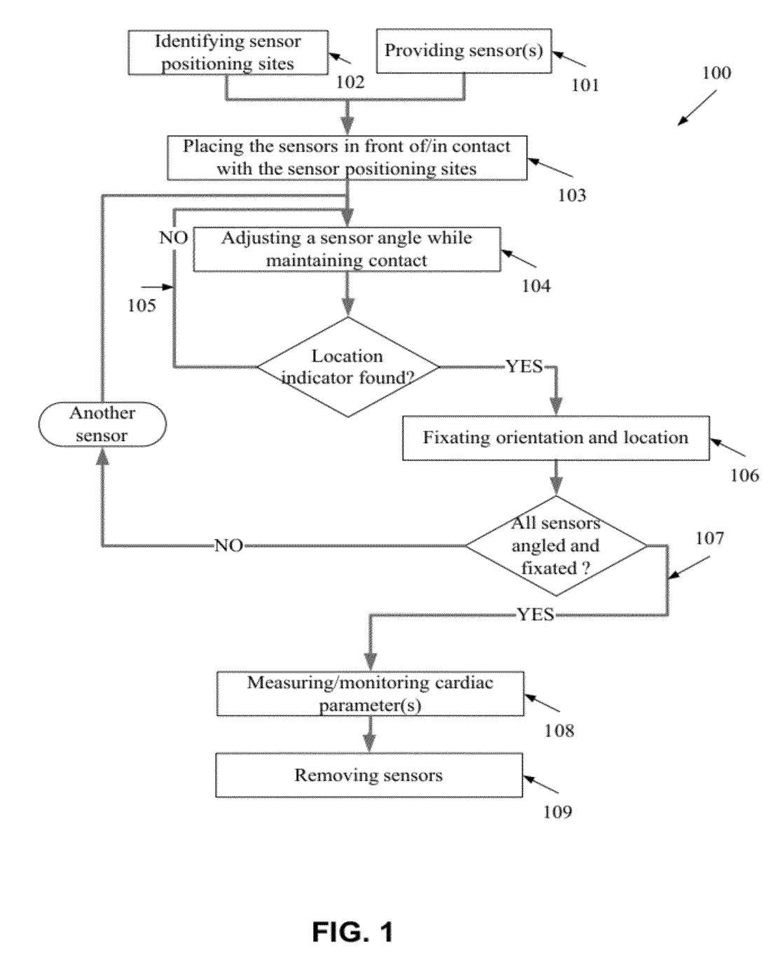

Reference is now made to FIG. 1, which is a flowchart 100 of a method of aiming sensors, such as one or more ultrasound probes, for non invasive measurement of one or more cardiac parameters, according to some embodiments of the present invention.

First, as shown at 101, one or more sensors, such as ultrasonic probes, also known as transducers, are provided. Each one of the sensors (i.e. transducers) has a front side contact zone (i.e. an active face of the transducer), that is sized and shaped to be in contact with a sensor positioning site on the skin of a target patient. The sensors are optionally connected to a cardiac parameters monitoring and/measuring system, for example as described in International patent application publication NO. WO2008/050334, published on May 2, 2008, which is incorporated herein by reference.

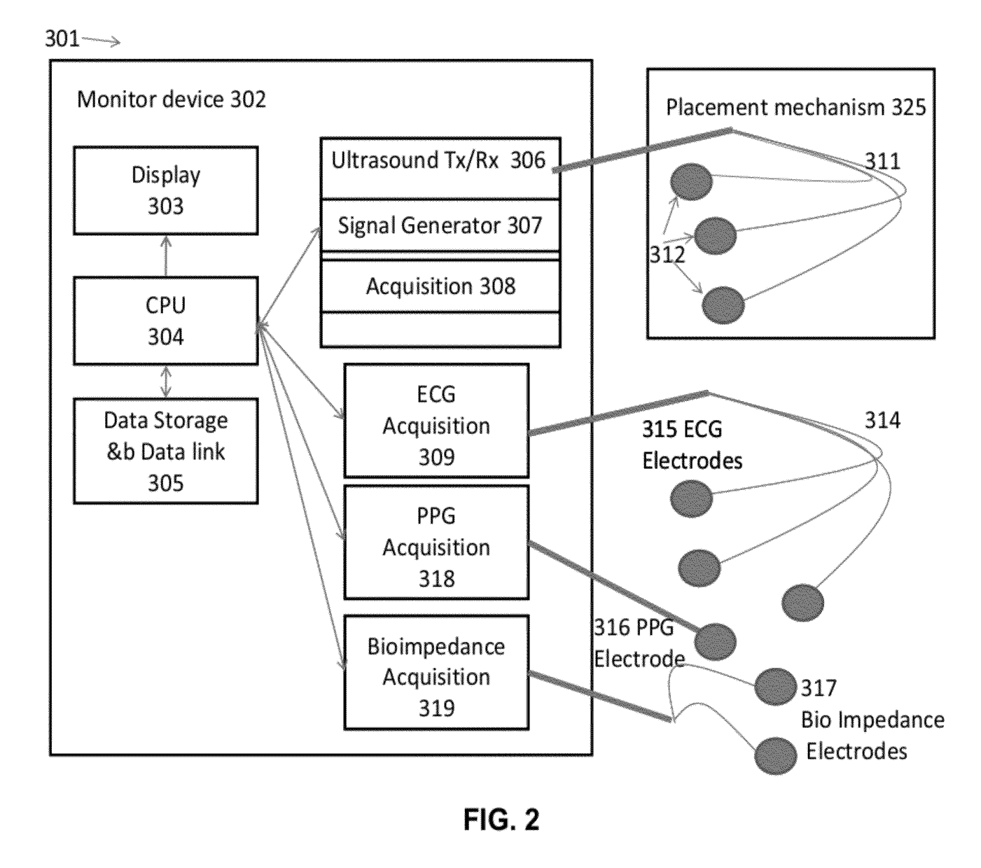

For example, reference is now made to FIG. 2 which is an exemplary monitoring system 301 including a monitor device 302, an ultrasound probe section 310 which includes a placement mechanism 325, and optionally an ECG probe section 313, and optionally a PPG sensor 316 and optionally Bioimpedance sensor 317 according to some embodiments of the present invention.

Optionally, a processing means such as a personal computer, a tablet, a laptop, and/or a digital signal processing (DSP) unit serves as the monitor device 302. The monitor device 302 includes a control and processing means unit 304, data storage and optional data link unit 305, an optional display unit 303, an ultrasound transmitter/receiver unit 306 that consists of an ultrasound signal generator 307, an acquisition unit 308, and an ECG acquisition unit 309, and optionally a PPG acquisition unit 318 and a Bioimpedance acquisition unit 319. In some embodiments, the ECG and/or PPG and/or bioimpedance and/or ultrasound sub-systems are separated, optionally external, modules which are controlled by CPU 304 and/or send data thereto. In a particular embodiment of the invention, the ultrasonic sub-system and the ECG are standard devices, such as an echocardiography device with an ECG detector built in and monitor device 302 processes data received therefrom. For example, a single transducer embodiment, as described below, may be integrated into an imaging sensor and/or system. Optionally, ultrasonic signal processing and/or ECG processing and/or PPG processing and/or Bioimpedance processing is performed by such separate units.

In some embodiments of the invention at least some ultrasonic processing is carried out by monitor device 302 to extract cardiac parameters, for example one or more of determination of the relative locations of chest transducers, Doppler signal extraction, processing generation of spatial map (if any) with attribution of Doppler signals, measurement of S′, E′, and/or E, and/or calculation of the ratio E/E′, for example as further described below.

In an exemplary embodiment an ultrasonic application section 310 is provided with one or more transducers, for example, 3 ultrasound transducers 312 that are connected to ultrasound Tx/Rx unit 306 by one or more leads 311. The position and/or orientation (i.e. the pointing angle) of the one or more transducers are set by the placement mechanism 325, for example as further described below.

Optionally, the leads comprise cables for transmission and/or reception and/or control lines. The transmission and reception cables are optionally made of either double-shielded coaxial cable or twisted pairs or any other type of wiring suitable for ultrasound operational frequencies.

In an exemplary embodiment of the invention, the transducers comprise separate transmitters and receivers (e.g., one or more of each). Optionally, at least some of the transducers act as both receivers and transmitters. Optionally, the transducers are transducers which provide a single beam (optionally using a shaping or a lens to shape the beam). The transducers 312 are optionally single element transducers 312. Optionally, one or more of the transducers 312 is an array transducer 312, for example, a phased array (such as annular or linear) element.

Optionally, the ultrasound transducers 312 are formed of PZT, cMUT or any other suitable material for Doppler ultrasound transmission and/or reception. In exemplary embodiments of the invention, the contact surface between the transducer's face and the skin is less than 1.5 cm in maximum dimension (e.g., 1.8 cm²), so that the transducer fits in the interspaces between the ribs. One or more of the transducers may exhibit a wide beam profile, for example as described below. Optionally, broad beams are provided using spherically shaped transducers or by using designated lenses to diverge the ultrasonic beam. Optionally, the beam is not circular in cross-section, for example, being elliptical with a height-width ration of between 1:1.2 and 1:6, or intermediate values, such as 1:2 or 1:4.5). Optionally, the beam is steered, for example, by rotation, mechanically and/or electronically.

In an exemplary embodiment of the invention, the ultrasound transducers and/or leads are reusable and mechanically durable. Optionally, the transducers are provided with a disposable covering, for example, a means for attachment to the chest. Optionally, the means provides good acoustic contact, for example, including gel or a matching layer and is sized to match inter-rib spaces. Optionally, the means includes a lockable joint or a plastically deformable attachment, for example a chunk of putty, to allow controlling, angling and fixing transducer orientation and/or distance between transducers. Optionally, the means includes a wireless transmitter. Optionally, the means includes ECG sensors and/or PPG sensors and/or Bioimpedance sensors and/or places therefore. Optionally, the means is a vest or a halter.

In an exemplary embodiment of the invention, one or more ECG leads 314 connect ECG acquisition electronics 309 to one or more ECG electrodes 315. In an exemplary embodiment of the invention, ECG signals are used to arrange data in a temporal manner, for example as described below or similarly using PPG signals and/or bioimpedance signals. In one example, QRS complexes are used to time onset of electrical systole, thus allowing for proper identification of subsequent Doppler derived signals from mechanical systole (S′, LVOT flow velocity envelope, VTI, SV) and diastole (E, E′, A, A′). This analysis may be useful in analyzing data from subjects with frequent extra systoles (premature ventricular beats and premature atrial beats), and irregular heart rhythms (e.g., as caused by atrial fibrillation). In an exemplary embodiment of the invention, the monitoring device 302 is used to detect an actual effect of arrhythmia. Optionally, an ECG trace and parameters as described herein are shown together, for example, in an ECG trace. Optionally, one or more of the parameters as described herein are fed in a continuous manner into an ECG machine or a stress test monitor.

Depending on the application, ECG signals may be used merely for detecting the beat-to-beat interval, For example, three ECG electrodes may be used for Lead II ECG signal.

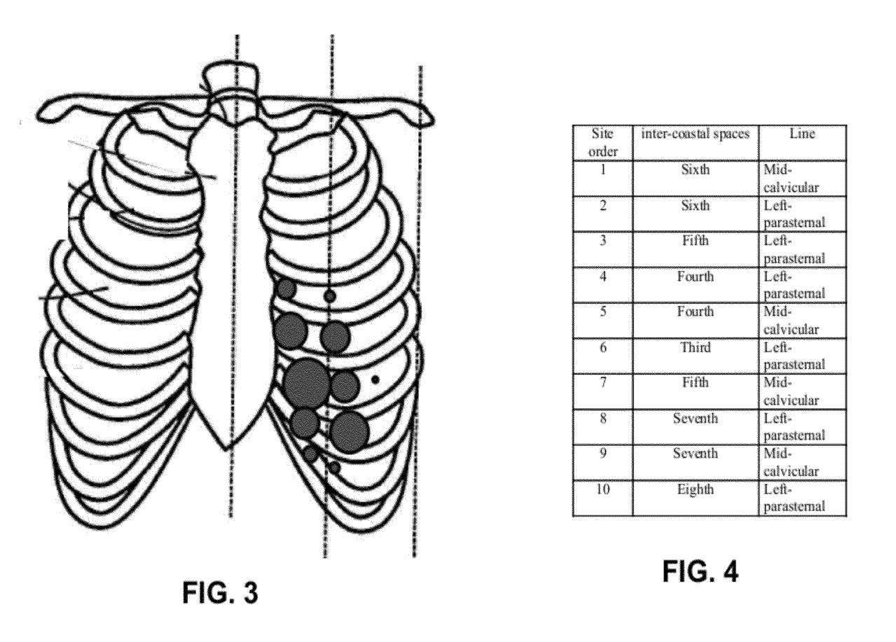

Reference is now made, once again, to FIG. 1. Optionally, as shown at 102, a plurality of sensor positioning sites are identified, for example mapped, on the chest of the patient. For example, the sensor positioning sites depicted in FIG. 3 were found by the inventors during a clinical study. The size of each dot is indicative of the likelihood to be an acceptable for sensor positioning as found in a clinical study. Note that larger circles mean higher likelihood, for example based on the number of reoccurrences in patients for the respective site. For example, FIG. 3 depicts the thorax and exemplary sensor positioning sites which are marked thereon (circles). These sensor positioning sites are selected for measuring cardiac parameters when the patient is in a certain position, for example in a supine position, for obtaining optimal views at mitral annulus and transmitral inflow. These sensor positioning sites are relatively flat for most patients regardless of their gender and/or age. Optionally, acoustic coupling gel is applied on the sensor positioning sites and/or on top of the front side contact zone of the sensors before the attachment of the sensors to the sensor positioning sites. Optionally, the sensor positioning sites are shaved before the attachment of the sensors thereto.

According to some embodiments of the present invention, an echocardiography imaging probe is used to identify and mark the sensor positioning sites. In such an embodiment, the adjustment of the signal capture angle of the sensor(s) may be performed using echocardiography imaging.

First, the echocardiography imaging probe is moved along the intercostal spaces while aiming the probe toward the mitral valve. The moving is performed until a four chambers view, closest as possible to an apical view, is identified.

Optionally, the output of the echocardiography imaging probe is analyzed with a Doppler module, such as the monitoring device (numeral 302 in FIG. 2). The output is optionally a Doppler ultrasound trace/signal, such as a time velocity Doppler trace. For example as described in International patent application publication NO. WO2008/050334, published on May 2, 2008, which is incorporated herein by reference. In such an embodiment, the operator places the echocardiography imaging probe at one of the positioning sites, for example the positioning sites depicted in FIG. 3, and places the imaging sample between the mitral leaflets and records a Doppler trace of a transmitral flow to verify the achieving of a transmitral blood flow signal (E and A waves) pattern.

Additionally or alternately, the operator places the echocardiography imaging sample at the septal side of the mitral annulus and records a Doppler trace to verify the achieving of an early diastole tissue motion (E′ septal-wave) pattern. Additionally or alternately, the operator places the echocardiography imaging sample at the lateral side of the mitral annulus and records a Doppler trace verifying achieving E′ lateral wave pattern. Additionally or alternately, the operator is taking the echocardiography image in non-apical views and places the sample, at the anterior or inferior sides of the mitral annulus to record E′ anterior wave or E′ inferior wave, respectively. At first glance the procedure described above seems similar to what is used in conventional tissue Doppler imaging (TDI). However, unlike common TDI where images are taken in a 4 chambers apical view, when the imaging probe is positioned at the heart apex, here the probe positioning sites are spread over the chest in non-standard views. This may require from the operator to identify a portion of the heart in non-familiar views.

Now, the location is marked on the skin of the patient, for example using a marker. Optionally, the angle and direction of the echocardiography imaging probe is also marked, for example marked with a marker, saved or logged in the memory or documented by the operator. Now, the echocardiography imaging probe is moved to another location aiming towards the mitral valve and the operator repeats the above described marking actions.

According to some embodiment of the present invention, the plurality of sensor positioning sites are identified using an ultrasound transducer, based on the analysis of tissue and blood Doppler signals, optionally as defined in WO2008/050334, published on May 2, 2008, which is incorporated herein by reference.

First, the device is switched to a setup.

Now, one of the sensors, for example an ultrasound transducer, is moved along an intercostal space, in proximity to one of the sensor positioning sites. The ultrasound transducer is optionally moved from one sensor positioning site to another, optionally in a predefined order, for example according to the order described in the table depicted in FIG. 4. In some patients other sites may also be checked.

The ultrasound transducer is optionally aimed toward the left ventricle where the aiming ranges from up-left to up-right and between 0 and 40 degrees. The sensor angled to increase a signal based location indication, which is optionally displayed on screen display and/or LED indicator which provide a real-time feedback and signal quality rating. The signal based location indication may comprise a number of interim signal based location indications which are indicative of a fulfillment of condition that is required for the acquisition of the signal based location indication. The signal based location indication is verified, optionally visually, for example when a continuous representing line appears on the screen display. Now, the operator can mark the location of the ultrasound transducer on chest, as a sensor positioning site, for example using a marker. The operator optionally documents the angle and the direction of the ultrasound transducer. Now, another sensor positioning site is identified by repeating the above functions. The process repeats until sufficient sensor positioning sites are found, for example three.

Now, as shown at 103, each sensor is attached to a body of target patient so that the front side contact zone thereof is on touch with one of a plurality of sensor positioning sites. Optionally, the sensor positioning sites are on the chest of the target patient, between the third and the eighth intercostal spaces (ICS) and between left to the para-sternal line and mid-clavicular lines (depicted in FIG. 3). In case of patients with a heart located in the right side of the chest, all positioning named in the left side will be named in the right side.

Then, as shown at 104, the signal capture angle of each sensor is adjusted in relation to the body of the patient while the contact between the front side contact zone of the sensor and the sensor positioning site is maintained. It should be noted that the sensor 201 may be one of a plurality of sensors in an array of sensors in a device (not shown) for a non invasive measurement of one or more cardiac parameters.

As shown at 105, the signal capture angle of the sensors is adjusted, optionally sequentially or simultaneously, until a signal based location indication such as a repetitive pattern of one or more cardiac parameters, is identified, for example as described below.

After the identification of the signal based location indication in one sensor either automatically by an analysis module or manually by an operator, the one or more sensors are fixated in the respective signal capture angle, while maintaining the front side contact zone in contact with the target area, as shown at 106. The process depicted by blocks 104-106 is repeated until all of the sensors are fixated, as shown at 107.

It should be noted that the fixating may be performed before all the sensors are angled, for example after the angling of each sensor separately.

By fixating sensors, such as ultrasound transducers, Doppler quantitative measurements may be reproduced in an accurate manner. In particular, the inter-observer and intra-observer variability is reduced from the order of 10-20% or even more when no fixating is performed. This provides a practical solution for a constant monitoring of gradual changes as it allows repeating the recording under the same conditions by fixating the sensors at exactly the same position and orientation during the monitoring. Moreover, the aforementioned positioning and angling are not susceptible to deviations and/or errors of a holding operator. In addition, the sensors are placed and fixated by the same placement mechanism and therefore do not yield different measurements as an outcome of different placement practices which are performed by different operators and institutions or the same operator in following examinations.

Reference is now made to a number of mechanisms that allow angling sensor(s) while maintaining front side contact zone(s) thereof in contact with target area(s) and fixating the sensor(s) when it is located in a desired position and orientation, for example when a signal based location indication is identified. These mechanisms are also defined to allow the fixating of the sensor(s).

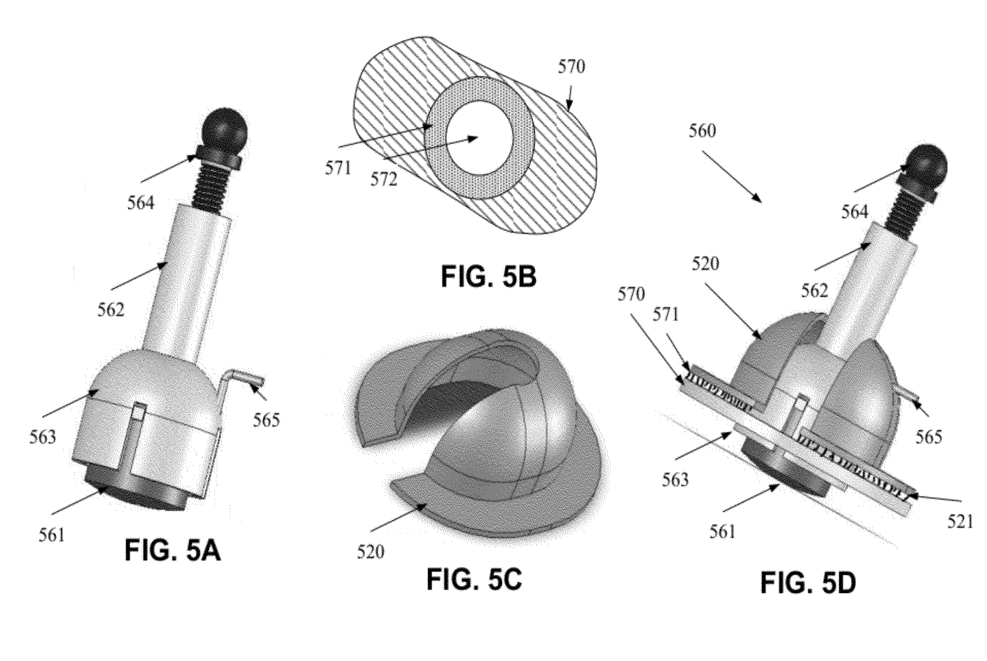

For example, reference is now also made to FIGS. 5A-5C, which are schematic illustration of components of a placement mechanism, for example as depicted in FIG. 5D, according to some embodiments of the present invention. FIG. 5A depicts an exemplary sensor 561, such as an ultrasonic transducer and an exemplary angling unit 560 which includes a hollow angling handle 562 having a housing 563, optionally hemispherical, for supporting the exemplary sensor 561, an elevation mechanism 564, such as a fastening element with a spiral grooved shaft which pushes and pulls the sensor 561 in and/or out of the housing 563, and optionally a locking element. For clarity, numeral 565 depicts a transducer cable that allows connecting the exemplary sensor 561 to a receiver. Reference is also made to FIG. 5B which is a schematic illustration of a label with a coating of adhesive on its back side, referred to herein as a sticker 570, having an opening 572 and an exemplary fastening element 571, optionally circular, around the opening 572. The fastening element 571 is optionally an interlocking nylon strip, for example with loops or hooks, such as a Velcro™ layer. Optionally, the sticker 570 serves as an ECG electrode. In such embodiments, the sticker optionally includes a dielectric contact site connected to a main unit so that at least LEAD II ECG signal is collected. Optionally additional ECG electrodes are separately used, in similar placement mechanisms or individually, for example three or more.

Optionally the sticker 570 comes with different sizes and shapes to fit broad range of chest anatomies/structures as may vary between subjects due to but not limited to gender, weight, height etc. In some embodiments one or more stickers (not shown) with different shape and larger adhesive area may be put on to increase fastening and stability of 560 relative to the skin.

Reference is also made to FIG. 5C which is a schematic illustration of a cover 520 having an exemplary fastening element 521 (shown in FIG. 5D) which is set to couple with the exemplary fastening element 571, such as an interlocking nylon strip, for example with loops or hooks, such as a complementary Velcro™ layer. FIG. 5D depicts how the cover 520 is coupled to the sticker 570 in manner that confines the movement of the hollow angling handle 562. When the sticker 570 is adhered to the skin of a patient, the confined hollow angling handle 562 and the sensor 561 are firmly attached to the monitored/analyzed patient. Optionally, as shown at FIG. 5C, the cover 520 has a hemispherical shape that allows angling the hollow angling handle 562 while maintaining contact between the front side contact zone of the sensor 561 and the sensor positioning site.

In use, the sensor 561 may be attached and titled, for example as follow:
1. Residues of gel at and near the respective sensor positioning site are removed.
2. The back side (adhesive) of sticker 570 is attached to the patient's skin so that a marker of the sensor positioning site is at the opening 572. The sticker 570 orientation may be chosen so that the long side thereof does not interfere with other attachment means.
3. An acoustic coupling gel is applied on the front side contact zone and/or directly onto the patient's skin, inside the opening 572.
4. A sensor, for example an ultrasonic transducer, such as 561, is placed at the center of the sticker's opening.
5. Using handle 562 the sensor 561 is aimed towards the left ventricle—tilted between up-left and up-right, between 0 and 40 degree until a signal based location indication is identified.
6. The position and orientation of the sensor is fixated using the locking element
7. The quality of a signal based location indication, such as described below, is verified.
8. If required, the elevation mechanism 564 is used to push the sensor 561 towards the body to increase acoustic contact.
9. If required a sticker with larger adhesive area is used to fix and hold the sensor 561.
10. If required the cover 520 is released and bullets 5-7 are repeated until the quality of the signal based location indication is acceptable.

Optionally, the action depicted in blocks 102, 103, and 104 are preformed sequentially per sensor 201. In such an embodiment, a sensor positioning site is identified, than a front side contact zone of a sensor, such as 201, is attached to the identified sensor positioning site and tilted while the contact between the front side contact zone and the sensor positioning site is maintained.

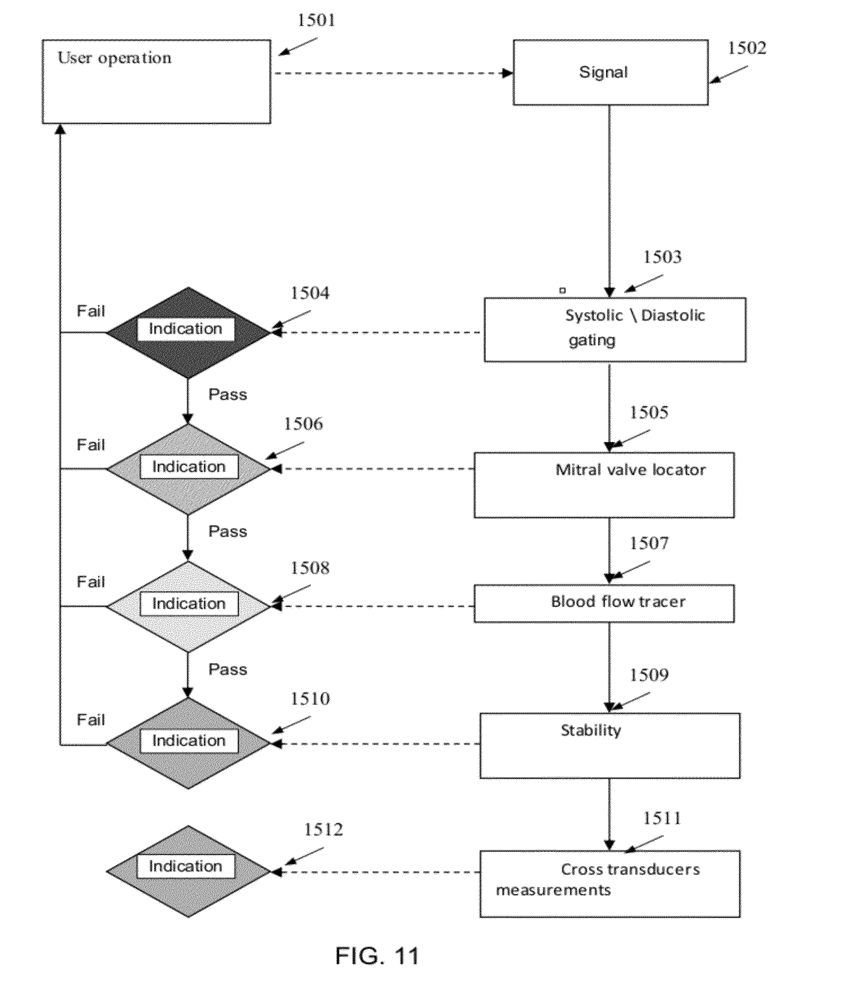

Optionally, the angling handle 562 is connected to a robotic arm and/or to actuating means which facilitate an automatic aiming of the sensor 561. In such an embodiment, the angling of the sensor 561 may be performed according to an automatic analysis of signal based location indications which are detected by analyzing the outputs of the sensor 561, for example as described with regard to FIG. 11. Optionally, the angling handle 562 is a manual handle which facilitates an operator to aim of the sensor 561.

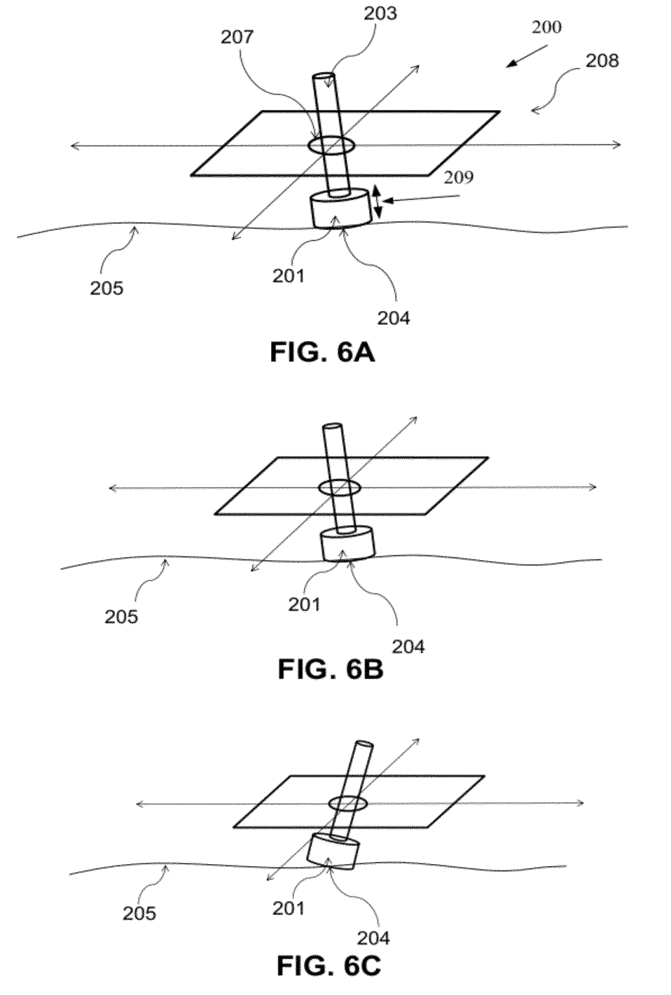

Reference is now made to another example. FIG. 6A is a schematic illustration of a placement mechanism 200 that allows adjusting the signal capture angle of a sensor 201 for non invasive measurement of cardiac parameter(s), such as an ultrasound transducer, according to some embodiments of the present invention. The sensor 201 is connected to a first end of a angling handle 203 which is part of an exemplary angling unit. For angling the sensor, the angling handle 203 is inclined while the first end is used as an anchoring point above the sensor head 201 at a point of contact 204 with the skin of the patient 205 at a sensor positioning site. Such angling may be performed by sliding a bounding element 207, such as a plate with an opening or a ring, in parallel to a sliding plane 208 which divides, for example bisects the angling handle 203. The bounding element 207 is optionally a board that slides in a sliding mechanism. The bounding element 207 angles the angling handle 203 so that the sensor 201 which is connected thereto is tilted while it remains in contact with the sensor positioning site 204, for example as shown at FIGS. 6B and 6C.

For example, the attachment and angling is performed as follows:
1. Residues of gel at and near the qualified sensor positioning site are removed.
2. The front side contact zone of the sensor 201 is attached to the skin 205. Optionally, the attachment is performed by applying acoustic coupling gel on the face of the sensor 201 and, while the sensor 201 is held at the sensor positioning site, the angling handle 203 attaches a sticker to skin. Optionally, the attachment is performed by placing the angling handle 203 in a bounding element 207, applying acoustic coupling gel on the face of the sensor 201, and attaching a sticker to the skin so that the sensor 201 is placed at the qualified site mark.
3. The sensor is aimed towards the left ventricle, where the angling ranges from up-left to up-right, from 0 to 40 degrees, until a quality of a signal based location indication is acceptable.
4. The position and orientation of the sensor 201 is fixed by locking the position of the slider plane 208 and at the same time the bearing of 203 relative to the slider plane.
5. The quality of the signal based location indication when the sensor is at the fixed position and orientation is verified.
6. If required, an elevation mechanism is used to push the sensor 201 towards the sensor positioning site to increase acoustic contact. Numeral 209 is indicative of the push/pull axis.
7. If required a sticker with larger adhesive area is used to fix and hold the sensor 201.
8. If required, the angling handle 203 and slider plane are released and bullets 3-5 are repeated until the quality of the signal based location indication is acceptable.

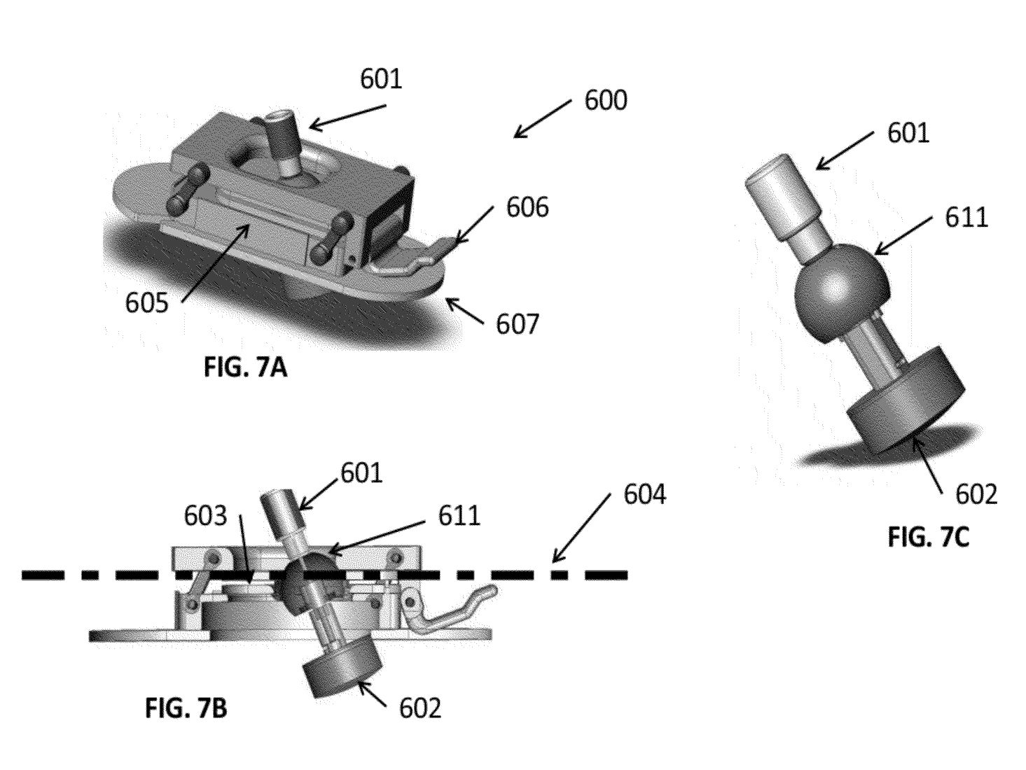

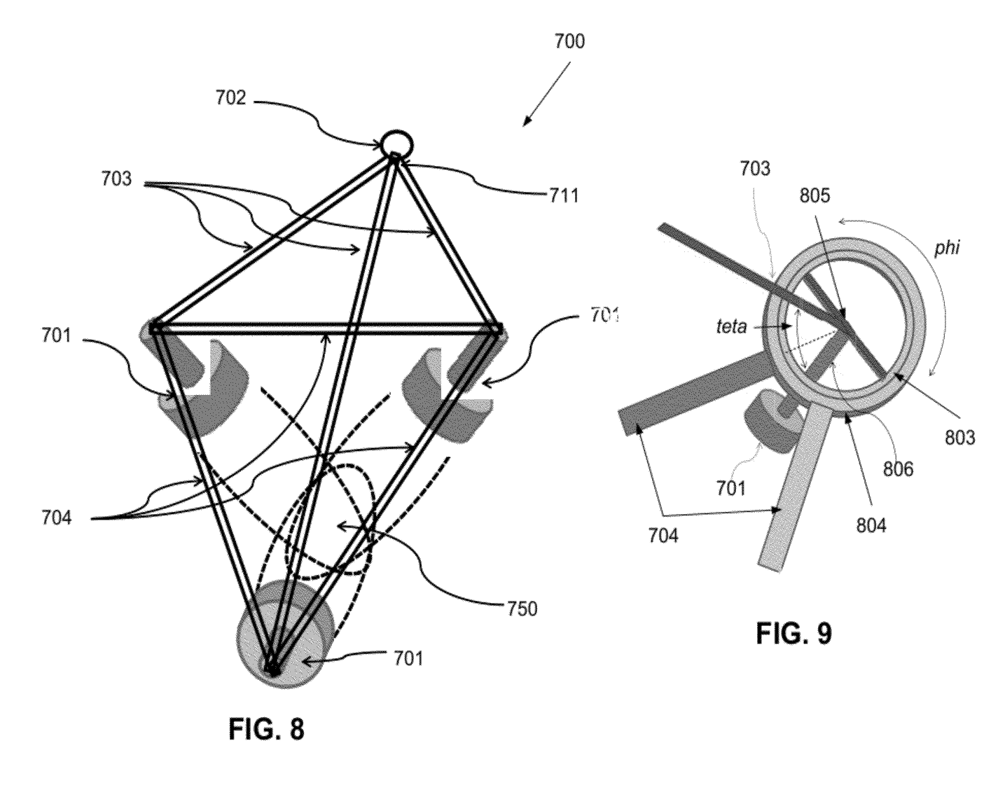

Reference is now made to a placement mechanism that angles a sensor as depicted in FIGS. 7A-7C. FIG. 7A depicts a placement mechanism 600 that angles a sensor 602 (shown in FIG. 7B which is a sectional schematic illustration of the placement mechanism 600) while it remains in contact with a sensor positioning site.

The placement mechanism 600 includes an exemplary angling unit with a plate 603 that is moved in parallel to a sliding plane 604 (shown in FIG. 7B), optionally in circles. The plate 603 is optionally connected to a frame 605 that rotates, optionally manually using a handle 601, above a pedestal, such as a sticker 607. The rotation slides the plate 603 in parallel to the sliding plane 604, angling the sensor 602 by manipulating a titling handle 601 that is connected thereto. The angling handle 601 optionally comprises a hemispherical or a spherical element (i.e. a ball bearing) 611 that allows angling the angling shaft 601 in relation to the sliding plane 604 using a ball bearing contact. For example, FIG. 7C depicts such an angling shaft 601. The titling handle 601 is optionally hollow to allow adding an elevation mechanism thereto, for example as described above. Handle 606 is used to lock the slider plane 603 and also to lock the ball bearing 611.

Optionally, the handles 606 and 601 are connected to a robotic arm and/or to actuating means which facilitate an automatic aiming of the sensor 602. In such an embodiment, the angling of the sensor 602 may be performed according to an automatic analysis of signal based location indications which are detected by analyzing the outputs of the sensor 602, for example as described with regard to FIG. 11.

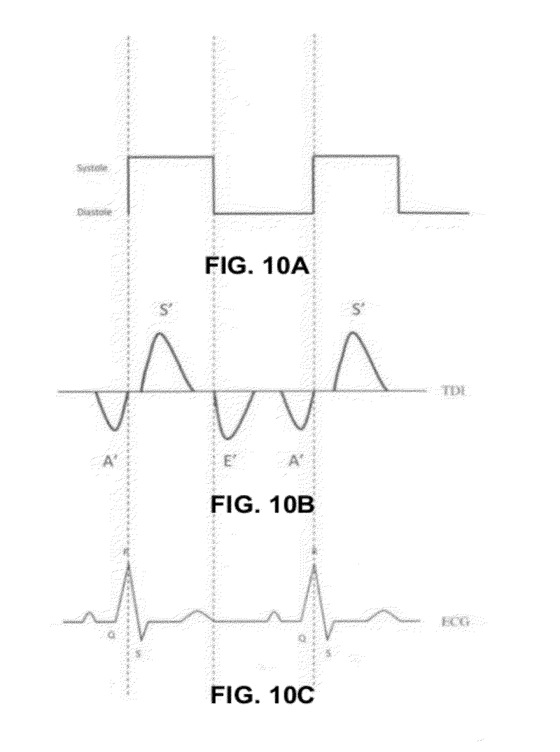

According to some embodiments of the present invention a stirring mechanism is use for adjusting the signal capture angle a plurality of sensors, such as ultrasound transducers, simultaneously. For example, reference is now made to FIG. 8, which is a schematic illustration of a stirring mechanism 700 that is connected for example to three sensors 701 and allows an operator to tilt them simultaneously by stirring a handle 702 located at a contact point, a stir junction 711, among three stir arms 703, optionally telescopic, which are connected to the three sensors 701. The length of the shafts 703 is adjusted to allow the simultaneous angling. The three stir arms 703 are connected to one another via supporting arms 704, optionally telescopic. Reference is also made to FIG. 9, which is a blow-up of a connection of arms in the stirring mechanism 700, according to some embodiments of the present invention. The supporting arms 704, 802 are optionally connected to one another using a rotary joint, such as concentric rings 803, 804, each attached to another supporting arm. The inner concentric ring 803 is connected to a sensor supporting bar 805 that is attached to an end of one of the stir arms 703 and to a angling handle 806 having one of the sensors 701 connected to its tip. The sensor supporting bar 805 is perpendicular to the stirring arm 703 that is connected thereto. This mechanism allows angling the sensor 701 by stirring the stir arm 703 (with other stir arms 703), an action that rolls the supporting bar 805 to tilt the angling handle 806 and the sensor 701 which is connected thereto. The stir arm 703 is connected to the sensor supporting bar 805 so that a phi rotation (in XY plane) of the stir arm 703 causes a phi rotation of the sensor 701 and a teta rotation of the stir arm 703 causes a teta rotation of the sensor 701. Optionally, the stir arms 703 and the angling handle 806 are set so that the location of the handle 702, the stir junction 711, is a reflection of an intersection 750 of the three ultrasonic beams which are emitted from the sensors 701. In other words, by moving the stir junction 711 it is possible to move the intersection 750 of the beams. All the sensors are moved simultaneously to change the location of the intersection 750 of the beams (the intersection is maintained). In such a manner, the mechanical device 700 may be used, optionally with external means, such as controllers, computing unit (CPU), and/or robotic arms, to facilitate a mathematically correct stirring of the sensors 701 wherein triangulation properties are maintained.

This stirring mechanism allows angling all three sensors 701, which are optionally broad beam transducers, to aim at the mitral leaflets area. This simplifies using a triangulation method to calculate the magnitude of peak velocity vector, its direction and its origin, see for example as described in International Patent Application, Pub. No. WO2008/050334, published on May 2, 2008, which is incorporated herein by reference. The triangulation equations have two solutions. If the three transducers 807 lie in a common X-Y plane, the origin of a velocity vector can be either in the positive Z values or its reflection in the -Z values. Because the sensors are place on the patient's chest it is easy to eliminate one of the solution by taking only the velocity vector originating from inside the body of the patient. With the above described apparatus the position of junction 711 has at any given time truly represents the reflection of beam cross-section 750 relative to the X-Y plane.

Reference is now made to a description of a process of angling a plurality of sensors simultaneously:
1. Residues of gel at and near the respective sensor positioning site are removed.
2. Three sensors, such as ultrasonic transducers, are attached to the skin of a patient, for example as described above.
3. Now, the stirring mechanism 700 is attached to each of the three sensors or otherwise in contact therewith.
4. This allows stirring all three transducers to obtain a signal based location indication with a quality above a certain level.
5. The position and orientation of the sensors is locked, for example as described above.
6. Optionally, the stirring mechanism is released and the quality of the signal based location indication is verified.
7. If required, elevation mechanisms are used to push separately the sensors 201 towards the sensor positioning site to increase acoustic contact.
8. If required a sticker with larger adhesive area is used to fix and hold the sensor 201.
9. If required the angling handle 701 is released and bullets 3-6 are repeated until the quality of the signal based location indication is acceptable.

It should be noted that the front side contact zones of the sensors are optionally maintained in contact with the sensor positioning sites during the stirring.

Optionally, the stirring mechanism is connected to a robotic arm and/or to actuating means which facilitate an automatic aiming of the sensors. In such an embodiment, the angling of the sensors 201 may be performed according to an automatic analysis of signal based location indications which are detected by analyzing the outputs of the sensors 201, for example as described with regard to FIG. 11. Optionally, the stirring mechanism is connected to a manual handle which facilitates an operator to aim of the sensors.

According to some embodiments of the present invention, the aimed and fixated sensor(s) are used for constant monitoring of cardiac parameters. Such a monitoring may be performed for periods of few minutes, few hours, and/or few days, or any intermediate or longer periods. As the sensors are fixated, the monitoring maybe performed in a hands free fashion.

Optionally, a single sensor is aimed and fixated, for example as described above. In such an embodiment, optionally after the device switches to a monitoring mode, cardiac parameters are measured and optionally shown graphically and/or numerically on a screen display. Additionally or alternatively, a monitoring module constantly monitors the one or more cardiac parameters.

Optionally, a plurality of sensors are aimed and fixated, for example as described above. In such an embodiment, optionally after the device switches to a monitoring mode, a short initial acquisition cycle is performed by a monitoring module that analyzes the outputs of the sensor. During an acquisition cycle each one of the sensors transmits a reference signal, for 30-60 seconds, while all the other sensors are in a receiving mode. This allows calculating the distance between the sensors based on time-of-flight, calculating position and angle of a blood velocity vector and a tissue velocity vector relative to the transmitting sensors and/or set monitoring mode parameters such as order of transmitting transducers, geometrical factors and/or signal quality, see for example as described in International Patent Application, Pub. No. WO2008/050334, published on May 2, 2008, which is incorporated herein by reference. At the end of the initial cycle, a notification is presented to the operator, for example a GO sign is displayed indicating normal mode of operation and/or a LED is energized. The monitoring module measures cardiac parameters which are optionally shown graphically and/or numerically on a screen display while the monitoring module constantly monitors the outputs of the sensors in a hands free fashion. Optionally, at any point, if the quality of the signals and/or measured quantities is reduced to an inadequate level, the monitoring module immediately prompts used and call for readjustment with instructions on possible causes of failure.

Reference is now made, once again, to FIG. 1. Now, after the orientation of the sensors has been adjusted and fixated, cardiac parameter(s) may be measured and/or monitored, as shown at 108.

The sensors may be removed any time, for example by removing attachment means, such as the aforementioned stickers and/or cover from the skin of the patient. In some embodiments, it is possible to temporary remove the sensor(s) while leaving anchoring elements such as sticker(s) so that reattachment to an exact position in front of sensor positioning site(s) is possible. Such an embodiment, may be used when the monitored patient switches beds and/or rooms, changes cloths, monitored in non continuous intervals, takes a shower, and the like.

As described above, the identification of a signal capture angle per sensor may be done by detecting signal based location indication by analyzing a Doppler echocardiography signal generated by the sensor. The signal based location indication provides a real time feedback to the operator on the quality of the transmitral flow of the tissue and valve Doppler echocardiography signals. It is mainly used to assist the operator during a setup mode, for example locating a sensor positioning site and during transducer positioning and angling.

The signal based location indication relies on anatomical and physiological considerations as well as known sensor, for example ultrasound transducer, design and performance to enhance only the relevant Doppler echocardiography signals that matter for the monitored performance.

Optionally, the direction of motion is taken into account. During a diastole, the transmitral blood flow, E wave, and a late diastole transmitral flow (A-wave), are positive, namely move towards the sensor, while tissue motion at mitral annulus is negative during diastole, E′ wave and A′ wave, and positive during the systole, systolic tissue motion (S′-wave), enhancement using forward or backwards Hilbert transform, or alternatively showing only positive, blood flow as opposed to conventional, for example in red and blue. The opening of mitral valve is in the direction of the transmitral blood flow, namely positive. The closing of the aortic valve is also positive.

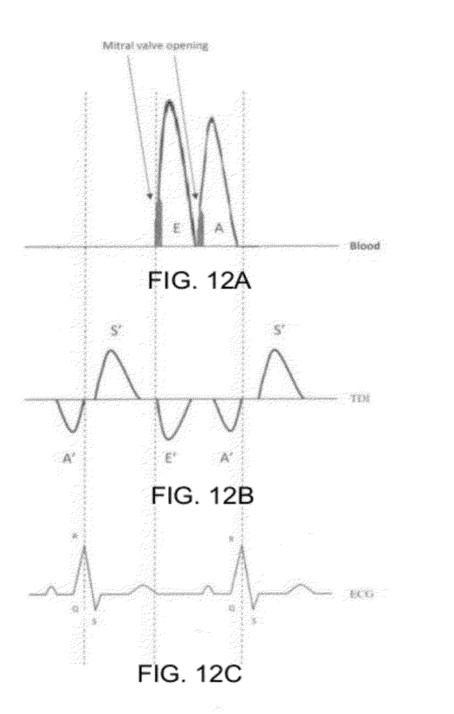

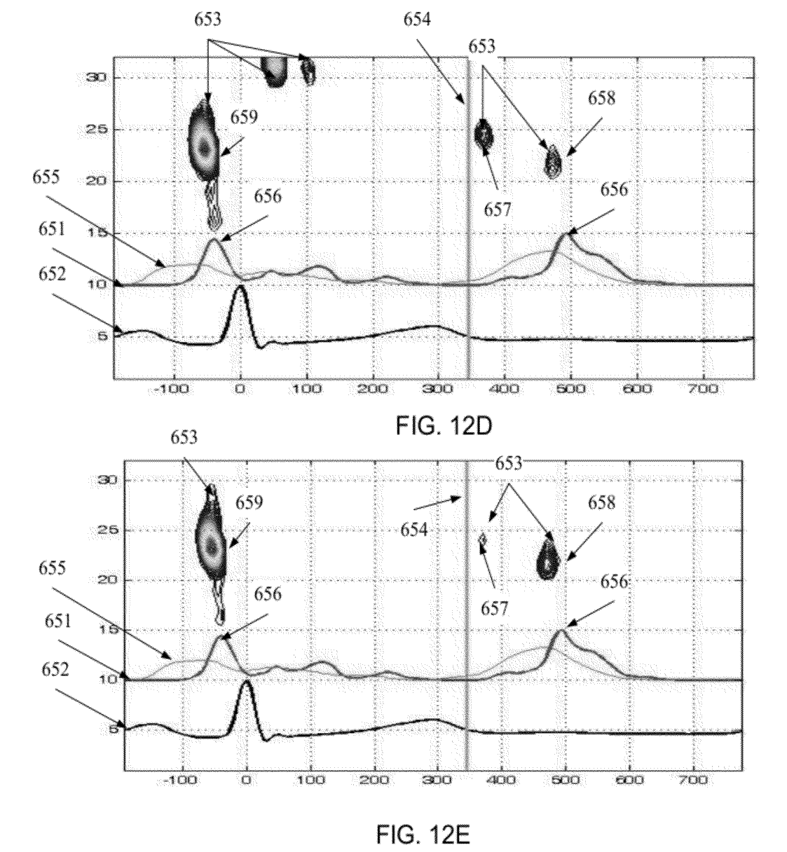

According to some embodiments of the present invention, mechanical concurrent events are taken into account during the detection of signal based location indication in the received Doppler echocardiography signal(s). During a diastole cycle, the blood accumulated in the left ventricle volume increases its volume so that E wave and E′ wave are physiologically conjugated, for example a positive transmitral inflow velocity is accompanied by negative motion of mitral annulus, for example see FIGS. 12A and 12B. The same is true for A wave and A′ wave, for example see FIGS. 12A and 12B. When tissue and blood Doppler are measured simultaneously, for example as described in International Patent Application, Pub. No. WO2008/050334, published on May 2, 2008, which is incorporated herein by reference, since E′ wave signal is typically 30 decibel (dB) stronger than that of E wave, E′ wave may be used for the timing of E wave. This allows performing measurements in the presence rhythm disturbance(s).

Another mechanical concurrent event is the opening of mitral valve at the beginning of diastole. This opening is followed by blood inflow and tissue motion. This may be identified by analyzing the simultaneous tissue and blood Doppler signals. A mitral valve Doppler signal is relatively strong in relation to a blood Doppler signal and is well defined in time (typically with an accuracy of tens of milliseconds) and in space (typically 1.5-2 cm) and therefore is easier to identify. See more about the identification of the opening of mitral valve below with reference to block 1505 in FIG. 11.

Another mechanical concurrent event is the location of the opening of mitral valve in relation to the closing of the aortic valve on the Doppler echocardiography signal. The time between these two events correspond to the iso-volumetric relaxation time.

According to some embodiments of the present invention, electro mechanical concurrent events are taken into account during the detection of signal based location indication from an electrocardiogram (ECG) signal. For example, the period of the QRS electrocardiogram (ECG) complex represents an onset of systole; see, for example, FIG. 10C. The end of a T wave corresponds to the end of systole and p wave is associated with an onset of atrial contraction. It should be noted that the R wave in the QRS complex is well defined in time and optionally used for Beat to Beat gating and/or for detecting the onset of systole.

Optionally, spatial relations are taken into account during the detection of a signal based location indication in the Doppler echocardiography signal. The maximal inflow velocity is close to the mitral valve leaflets. Optionally, identification of the location of the valves is used for finding the location of peak transmitral velocity (E). The level of the mitral annulus relative to the level of mitral valves depends on the line of sight from the front side contact zone of the sensor. For example, when the sensor is located to image apical and near apical views, the mitral valves are approximately at the same distance from the front side contact zone of the sensor (i.e. transducer) and the mitral annulus is approximately 1-2 centimeters (cm) beyond the mitral valve leaflets. When the sensor is placed to image near left para-sternal views, the anterior mitral valve (MV) leaflet can be 1-4 cm closer than the anterior MV leaflet on the received Doppler echocardiography signal. The mitral annulus lies oblique to the ultrasound beam at distances ranging from near (distance to anterior leaflet) to far (distance to inferior leaflet) leaflets. The aortic valve is located at the end of the left ventricle outflow tract (LVOT) and therefore from most views (in the sensor positioning sites) the mitral valve is closer to the transducer face than the aortic valve.

According to some embodiments of the present invention, signal based location indication is detected by combining information from the time, spatial and direction of motion dimensions as recorded in the Doppler echocardiography signal. For example, reference is now made to FIG. 11, which is a flowchart of a method for detecting signal based location indication indicative of a potential signal capture angle for a sensor, such as an ultrasound transducer, according to some embodiments of the present invention.

From the operator point of view, the following is performed for identifying a potential signal capture angle:

First, as described above and shown in 1501, the sensor(s), for example the transducer(s) are placed in front of and/or on sensor positioning site(s). Now, the sensor(s) are tilted so that its front side contact zone is aimed toward the heart. The angling is performed from up-right to up-left, between 0 to 40 degrees. This is performed until a Doppler echocardiography signal is received from the output of the sensor(s), as shown at 1502. At each angle, the presence or absence of a number of interim signal based interim indicators is detected in succession. Optionally, indications to these interim signal based interim indicators are presented to the operator. Optionally, a later stage may be achieved instantaneously with a former stage.

As shown at 1503, systole and\or diastole periods are gated. For example, the systolic and diastoles are determined based on an analysis of a tissue Doppler signal. A transition from systole to diastole corresponds to transition from positive to negative tissue Doppler velocities whereas transition from diastole to systole corresponds to transition from negative to positive tissue Doppler velocities, for example see FIGS. 10A and 10B. Optionally, an ECG signal is used to mark the start of systole by QRS detection, see for example, FIG. 10C. Optionally, an indication of the gating success is presented to the operator, as shown at 1504.

Now, as shown at 1505, the mitral valve is identified. Optionally, mitral valves' opening is timed and the distance to mitral valves, on the received Doppler echocardiography signal, is identified using a band pass filter for separating Doppler echocardiography signals having a positive velocity ranging between, for example, 15 cm/s and 25 cm/s and/or identifying a correlation to tissue signals which follow a mitral valve signal see FIGS. 12A-12C. These diastole tissue velocities are necessary negative (E′ wave and A′ wave) and can be enhanced using a band pass filter between −2 cm/s and −20 cm/s. Optionally, an indication of the detection of a mitral valve opening is presented to the operator, as shown at 1506.

For example, reference is now made to FIG. 12D, which is a schematic illustration of traces which are extracted from one or more received Doppler echocardiography signals and an ECG signal or a processing thereof, according to some embodiments of the present invention. The display is for a single heartbeat. The traces 651 652 655 are depicted on a set of notches wherein X axis depicts time in milliseconds and Y axis depicts discrete points, range gates, in a range along multi radial of sensor data at which the received Doppler echocardiography signal is sampled.

Trace 652 is an ECG trace where its R peak represents zero time associated with onset of systole. As described above, the systole and the diastole are gated. Systole/diastole line 654 depicts such gating.

Potential mitral valve opening traces, for example as shown at 653, are generated according to a power spectrum density (PSD) of positive blood Doppler frequencies equivalent to a certain tissue velocity range, for example between +19 and +23 cm per second. A colour scale is used to depict the power density (from lower density (blue) to higher (Red). The reasoning for using this PSD presentation stem from the following properties associated with mitral valve opening:

a. blood enters the left ventrical in high velocities, via respective mitral valves that open with positive Doppler velocities which are typically higher than positive myocardial tissue velocities (typically less than +15 cm per second) thus myocardial tissue may be filtered; and

b. the intensity of signals intercepted from the mitral valve is much stronger than the signal intensity which is intercepted from the blood. By inspecting the PSD, blood signals may be filtered.

The Doppler echocardiography signal trace 651 is optionally the power spectral density (PSD) of negative (relaxing) tissue Doppler frequencies equivalent to −10-−3 cm per second, summed over a range of between 6 cm and 12.5 cm. The peaks indicated in 656 which are indicative of tissue movement induced by the transmitral blood inflow that is allowed by the opening of the mitral valve.

Optionally, the diastolic tissue Doppler signal trace 651 is weighted according to the mean value of time bins, for example 12, 16, or any other suitable number of time bins, for example using a Forward function. Trace 655, coloured in pink, is indicative of an outcome trace which is generated by weighting the Doppler echocardiography signal trace 651.

The closing of the aortic valve marks the end of systole and the opening of a mitral valve is at the beginning of diastole. The systole/diastole line 654 is optionally used to roughly identify transition from systole to diastole, and is used for eliminating potential mitral valve opening traces 653 which are identified in the systole, for example between the R wave of the ECG trace 652 and the systole/diastole line 654. The opening of a mitral valve is followed by blood inflow and tissue motion. A peak in the weighted Doppler echocardiography signal trace 656 is optionally used to eliminate false potential mitral valve opening traces from the options of 653. For example, eliminating all the potential mitral valve opening traces which are identified substantially before this peak, for example see 657 which is indicative of the aortic valve closing peak. While the first opening of the mitral valve 658, which is associated with early filling, is always present in many cases a second mitral valve opening corresponding to atrial contraction at the end of diastole is also present 659 (see left PSD peak centred at −70 ms in FIG. 12E). In the latter case the second mitral valve opening will appear just before the end of diastole. The period between the closing of the aortic valve event 657 which marks the end of systolic outflow, and the first opening of the mitral valve event 658 which marks the onset of diastolic inflow, is associated with the isovolumetric relaxation time. Thus, by analysing the aforementioned trace, the isovolumetric relaxation time may be estimated.

It should be noted that in the embodiments described above, the opening of the mitral valves is identified without imaging heart, based on Doppler echocardiography signal and ECG traces only. Optionally, the above traces are not presented to a user but rather analyzed to automatically identify the opening of the mitral valve.

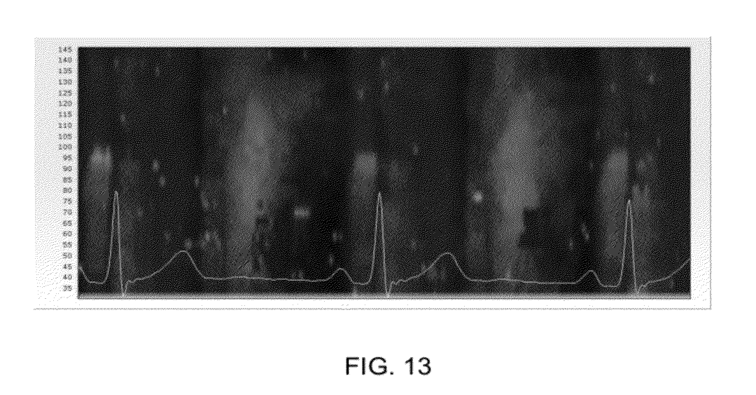

Now, as shown at 1507, the direction (polarity of Doppler velocity) of the blood flow is identified and a respective indication is optionally presented to the operator, for example as shown at 1508 and depicted in FIG. 13. Optionally, blood inflow maps are displayed. Optionally, the inflow maps are range-time maps, such as modified Doppler M-mode maps, for example where brightness level is indicative of velocity showing in Red only positive velocities, optionally greater than 30 cm/s, and negative velocities are not shown. The onset of a systole, determined using QRS detector and/or tissue gating, is marked as well as the onset of diastole. Optionally, color scale may be used to represent a real maximum positive velocity. Alternatively, a color may represent auto-correlation and/or energy of inflow velocity signal.

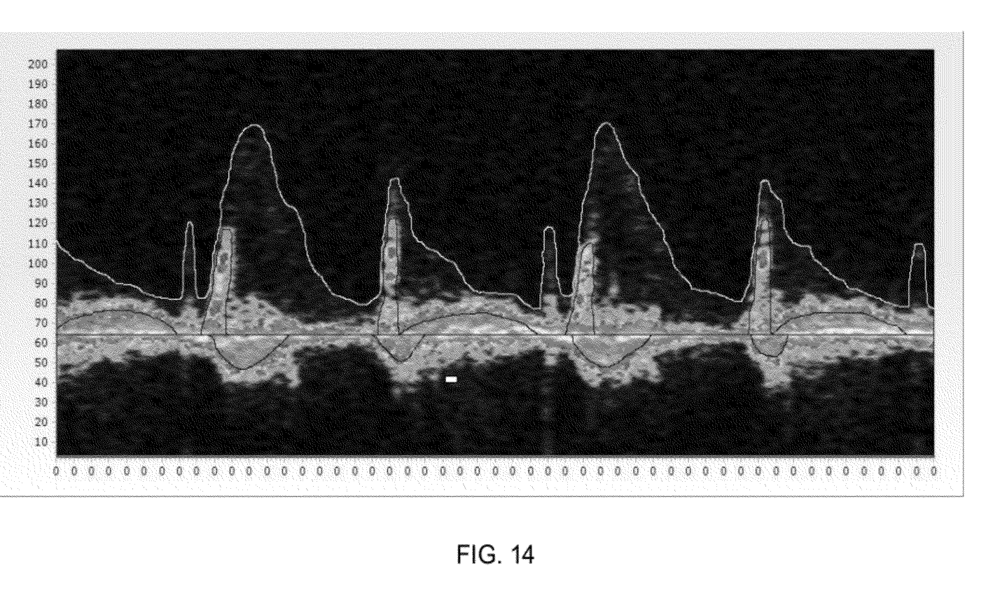

Optionally, enhanced Doppler traces of blood and tissue are displayed for any chosen range gate. The enhanced traces are optionally generated using a non linear (logarithmic) color scheme to show concurrent events of relatively strong tissue signals and relatively weak blood signals, for example see FIG. 14. Optionally, blood flow appear in bluish and cyan colors, tissue signals appear in magenta and white colours, valves appear in green and red colours, and background noise in black and/or dark blue. Optionally, the displayed gate is chosen automatically based on maximum signal intensity, maximum velocity, and/or near the mitral valve. In a preferred embodiment the edges of blood, tissue and valve signals are displayed in real time.

Optionally, the quality of blood signal is scored according to various parameters such as its highest velocity, its maximum energy and/or its pattern in time and space. This scoring may be automatic, for example by converting the values of the pixels in the image to values.

Now, as shown at 1509, the stability of the interim signal based location indications is verified, for example by beat to beat statistics, to quantify the signal quality over a period longer than a single respiration cycle, for example over 10 seconds. Optionally, a respective indication is optionally presented to the operator, for example as shown at 1510. This indication may be indicative that the location and the orientation of the respective sensor are adjusted to operational measuring of cardiac parameters.

Optionally, as described above, multiple sensors, for example transducers, may be used to acquire cross-channel signals, for example a signal received in channels corresponding to a sensor at various sites. Optionally, as shown at 1511, blocks 1501-1510 are repeated for each sensor. Optionally, if blocks 1501-1510 are not completed for a certain sensor at a certain sensor positioning site, the certain sensor is moved to another sensor positioning site.

If not all four stages are achieved, the sensor is moved to another sensor positioning site and the operator tries to perform the four stages yet again. Optionally, if an M-Mode view of blood flow is acquired, for example see FIG. 13, the operator tries to acquire the brightest and widest positive flow during a diastole.

After all the sensors have located and tilted, cross transducers parameters may be extracted, as shown at 1512. Optionally, when a plurality of sensors are used, the signal capture angle of the sensors is further adjusted, for example optimized, to acquire better cross-channel signals, for example the receiving signal in other channels corresponds to sensors at different sites. Now, as shown at 1513, an indication of a process completion may be presented to the operator.

Optionally, the aforementioned indications are generated by an array of indicators, such as colourful LEDS, a speaker, and/or a display, such as an LCD.

Now, the operator may fix the orientation and location of the sensor(s), for example as described above.

Optionally, if M-Mode view of blood flow is achieved, for example as shown at FIG. 13, a bright and wide image (as much as possible) of a positive flow, during a diastole is acquired.

Now, the signal capture angle and position of the sensor is fixated.

Optionally, as shown at 108, the outputs of the sensors, for example Doppler ultrasound signals, are analyzed to measure non invasively one or more cardiac parameters. The parameters are optionally cardiac parameters, for example one or more of the following: early diastole transmitral flow velocity (E), myocardial wall velocity during early diastole (E′), myocardial wall velocity during early systole (s′), a late diastole transmitral flow velocity (A), tissue motion velocity (A′), the blood and tissue velocity ratio E/E′, the blood and blood velocity ratio E/A, tissue to tissue velocity ratio (E′/S′), left ventricular outflow tract (LVOT), velocity time integral (VTI (S′) and VTI (E+A), stroke volume (SV), Cardiac output (CO) and/or heart rate (HR). The extracted values may be presented in raw form, averaged and/or otherwise filtered, for example, by smoothing, or by binning to match different cardiac rhythm morphologies.

Optionally, a statistical analysis of the cardiac parameters is calculated. For example, the cardiac parameter(s) includes VTI (E+A) diastolic inflow that follows the stroke volume (SV), and the statistical analysis includes calculating stroke volume variability (SVV), a predictor for fluid responsiveness in ventilated/anesthetized patients, according to a pulse-contour analysis and aortic Doppler for beat-by-beat measurement. The SVV is optionally calculated from the stroke volume values over a few respiration cycles, optionally 6 seconds each, where a value reflects a ratio between minimal and maximal SV in percentage.

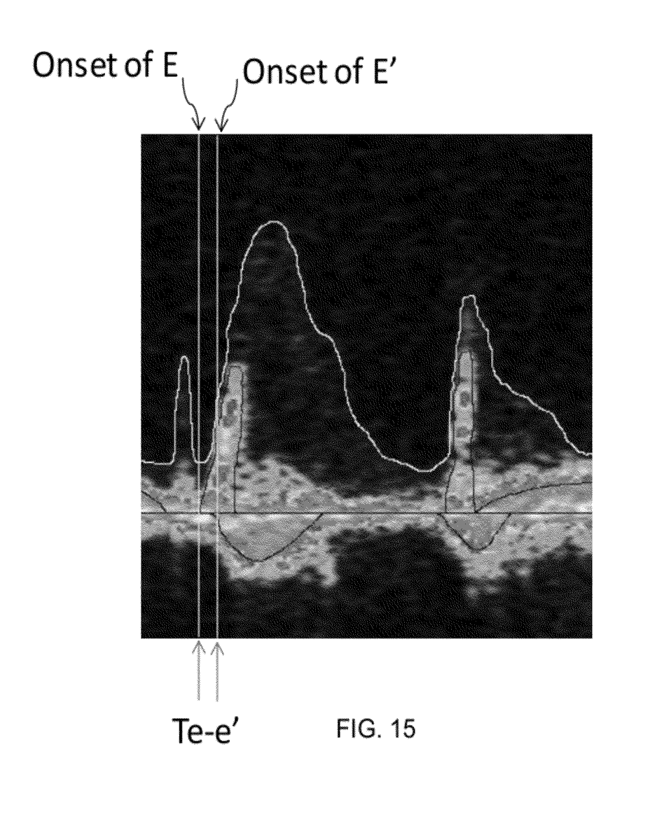

Optionally, the measured cardiac parameters include timing between events. Such cardiac parameters may include isovolumetric relaxation time (IVRT), isovolumetric contraction time (IVCT), and/or a delay time between the onset of E and the onset of E′, for example see Te-e′ in FIG. 15. In TDI quantitative measurement of E/E′, the heart rate is assumed to be steady between measurements of E and E′ in order for the ratio E/E′ to be meaningful. In pathologies of rhythm disturbance, conventional TDI is inadequate because blood and tissue velocities are not recorded for the same heartbeat and the ratio E/E′ is meaningless. The ASE recommendations teach on a very promising method to identify elevated filling pressures by looking at the time delay (Te-e′) between the onset of E and E′. According to a series of studies instructed on the relation between Te-e′ and the relaxation coefficient of the myocardial wall (tau) and further showed very good correlation between the inverse of Te-e′ and filling pressure. It should be noted that for certain subgroups of patients, such a correlation is superior in relation to an E/E′ ratio correlation. However, in practice this measurement is very complicated because with conventional echo scanners the timing of these events (the onset of E and the onset of E′) can only be determined relative to the ECG QRS complex. This is because conventional echo requires the user to change the mode from standard (blood) to tissue Doppler between measurements and simultaneous measurement of tissue and blood Doppler are not available.

A generic measurement that can be done in various ways is the measurement of isovolumetric contraction time (IVCT) and isovolumetric relaxation time (IVCT). The IVCT is the time between the closing of mitral valve (end of late transmitral flow) and the opening of the aortic valve (onset of systolic flow), whereas the IVRT is the time difference between the closing of the aortic valve and the opening of the mitral valve (onset of early diastole filling). These measurements, conventionally done in a 5 chambers views and placing the sample between the levels of the aortic and the mitral valves and looking at the timing of systolic outflow and diastole early and late inflow. Alternatively, the clicking (audio closing sound) of the valves can be used to identify the end of systole. Alternatively, the timing of the iso-volumetric periods can be measured directly from their recognized (sinus) pattern in the tissue Doppler traces.

Optionally, the measured cardiac parameters include indices which are calculated based on time measurements such as Tei index, which is an echocardiographic index of combined systolic and diastole function, calculated as isovolumetric relaxation time plus isovolumetric contraction time divided by ejection time.

Optionally, the measured cardiac parameters are calculated according to spatial information of the received signal(s), for example a distance between signal segments which are indicative of valve leaflets, a distance between signal segments which are indicative of mitral annulus, and/or a distance between signal segments which are indicative of a local peak velocity of blood.

Optionally, an ECG signal is simultaneously acquired with the Doppler ultrasound signals, such as time velocity Doppler traces.

Optionally, the readout of Doppler ultrasound signals is performed on-line by one or more software browsing tools.

Optionally, the attachment means which are described above, for example the sticker 570 and angling unit 560 are disposable. In such a manner, the sensors and the aforementioned placement mechanisms remain sterile. Optionally, the sensor 561 is also disposable.

According to some embodiments of the present invention, the sensors are wide ultrasound beam transducers. In such embodiments, the cardiac parameters may include measurement of bulk velocity of the mitral annulus. In an exemplary embodiment of the invention, the cardiac parameters are displayed numerically and graphically on a screen and/or stored in a storage unit. Optionally, the cardiac parameters are displayed continuously (or at any other rate as determined by the clinical staff), optionally, with no limitation on patient posture. As the measuring is performed noninvasively, it may be used for any desired time length with little or no fear of infection.

Optionally, the sensors are positioned on a patient and yield usable results without the intervention of an expert and/or without using an imaging step to position the system. In some embodiments, once the sensors are positioned and angled, even using an expert and or imaging for guidance, no expert is needed to maintain the system in a usable positioning, for appreciable periods of time. In some embodiments of present application, as described above, the angling of the sensors is done with the assistance of indications which are presented to the operator, for example whenever an interim signal based location indications are identified. Thus, the training of an operator placing the sensor may be easier than the training of an operator that has to place respective sensors in known placing methods.

As described above, the sensors may be used for acquiring tissue (i.e. muscle) signals and blood signals in a common cardiac cycle, possibly providing meaningful results even in arrhythmic patients or other patients with large beat-to-beat variability, for example as described in International Patent Application, Pub. No. WO2008/050334, published on May 2, 2008, which is incorporated herein by reference.

According to some embodiments of the present invention, the sensors are placed to monitor cardiac parameters for an Echo stress test. The Echo stress test is a good example in echocardiography where repeated measurements are needed. Because of its limitations only two echo measurements are usually done: one before the stress test (at rest); and one after the test (at stress). Immediately after the end of the stress protocol the patient goes off the treadmill and lies down on bed. This allows only a very short time window for the ultrasound operator to position the ultrasound probe and to take recordings before the patient is recovering from the stress state. This is one of the reasons why in echo stress testing a cardiologist looks only at the 2D image time loops, and qualitatively comparing stress and rest images looking for structural abnormalities.

Yet, recent studies have found that standard and tissue Doppler have great potential in echo stress testing. For example, according to these studies there are significant differences in the amount of change from “rest” to “stress” in the peak transmitral flow velocity (E), peak tissue velocity (E′), as well as in their ratio (E/E′). Moreover, the rest and stress values of these parameters and the pattern of their variations during the test are different between different groups of normal and abnormal patients. While evidently these findings potentially teach on superior way for early detection of ischemia, implementation of such method is practically difficult and cumbersome with the present art.

Usually, routine echo measurements are done in a set of conventional so called “views”, for example 2 chambers, 4 chambers and 5 chambers 2D images of the heart are all taken from an apical view. In practice, the operator usually places the probe somewhere at the mid-clavicular line and, while looking at the ultrasound 2D real time image the operator moves the imaging probe until it is closest to the cardiac apex. These standard views were originally selected to demonstrate the anatomy, function and dynamics of the heart. Consequently, they can be found in any echo textbook and guidelines (e.g. ASE guidelines). The standardization is crucial for various reasons:

a) to allow echo specialists to do the readings in off-line;

b) to allow qualitative and quantitative comparison between patients or between follow up measurement on the same patients;

c) to allow the clinical community to set norms for clinical parameters based on the standardized views; and in echo Doppler; and

d) to allow as much as possible unbiased measurement of Doppler indices.

This is based on a series of simplified assumptions about the geometrical relations between the angle of insonication (the propagation of ultrasound wave into the body is roughly described by a straight line connecting the face of the transducer and the target) and the vector of motion of a moving target (i.e. blood, tissue, valve etc.). For example, to measure the peak early transmitral velocity (E) the probe is placed in a 4 chambers' “apical view” (i.e. a cross section where four chambers left and right ventricles and associated atris are seen) and the sample is placed at the level and between the mitral leaflets. The underlying assumption is that the blood flowing into the heart through the mitral valve is generally directed towards the apex, and therefore the angle (teta) between the blood vector of velocity and the insonication line is very small. As a result the measured Doppler velocity, which is scaled by cos(teta), is very close to the real magnitude of the velocity vector.

According to ASE guidelines, a teta of less than 30 degrees is considered acceptable for quantitative measurements.

Clearly, this assumption is very rough since cos(30)=0.86 could lead to considerable underestimation of Doppler velocity by ˜14%. The situation is even more involved in tissue Doppler imaging since the actual motion of the myocardial wall may be composed of radial, longitudinal and twist movements, so it is not easy to define the exact angle of motion. In clinical practice, however, the ASE guidelines instruct how to measure the longitudinal component of the velocity of the mitral annulus motion (E′) and this is done from apical view.

Many of the standard measurements are done in apical view. However, in most cases it is technically impossible to obtain good apical views for patients in supine position. Therefore, most often (almost in all cases) patient are laid on their left side to allow better acoustic window for apical views. Therefore, critical care patients, in operating rooms and ICU, impose great challenge in echo cardiology because their supine position cannot be tolerated. By using the aforementioned methods and/or devices, sensors are fixated to the patient regardless of the patient position and therefore allow accurate determination of Doppler peak velocities in non-standard views—, for example in a supine position which is relevant in critical care patients.

It is expected that during the life of a patent maturing from this application many relevant systems and methods will be developed and the scope of the term signal, sensor and transducer is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A placement mechanism for placing a sensor for non invasive measurement of at least one parameter, comprising:
a angling unit which angles a sensor in relation to a sensor positioning site on a skin of a target patient in proximity to the ribs while maintaining a front side contact zone of said sensor in contact with said sensor positioning site; and
an attachment element for attaching said placement mechanism to a body of said target patient so that said front side contact zone being in contact with said sensor positioning site.

2. The placement mechanism of claim 1, further comprising an elevation element for adjusting the distance between said front side contact zone and said sensor positioning site.

3. The placement mechanism of claim 1, further comprising a label having a first fastening element on a front side and a coating of adhesive on a back side, said label being sized and shaped to be temporarily attached to said skin, at least partly around said sensor positioning site; said attachment element having a second fastening element configured for being detachably connected to said first fastening element.

4. The placement mechanism of claim 3, wherein said label has an opening to allow placing said front side contact zone in contact with said sensor positioning site.

5. The placement mechanism of claim 3, wherein said angling unit comprises a housing for supporting said sensor and a substantially hemispherical element forming a joint with said housing so as to allow angling said hemispherical housing with said sensor; wherein said second fastening element being connected to said hemispherical element.

6. The placement mechanism of claim 5, wherein said angling unit comprises a shaft connected to said housing; wherein said substantially hemispherical element has a slit for facilitating the angling of said shaft.

7. The placement mechanism of claim 1, further comprising at least one locking element for fixating said sensor in a signal capture angle while maintaining said front side contact zone in contact with said sensor positioning site.

8. The placement mechanism of claim 1, wherein said angling unit comprises a slidable element configured to be maneuvered in parallel to said a sliding plane so as to adjust a signal capture angle of said sensor in relation to said sliding plane while maintaining said front side contact zone in contact with said sensor positioning site.

9. The placement mechanism of claim 1, wherein said angling unit comprises actuation means for angling said sensor according to instructions from a monitoring device analyzing an output of said sensor.

10. A placement mechanism for placing a sensor for non invasive measurement of at least one parameter, comprising:
a angling unit which simultaneously angles a plurality of sensors in relation to a plurality of sensor positioning sites on a skin of a target patient, while maintaining a front side contact zone of each said sensor in contact with a respective said sensor positioning site; and
an attachment element for attaching said placement mechanism to a body of said target patient so that said front side contact zone being in contact with said sensor positioning site.

11. The placement mechanism of claim 10, wherein said angling unit comprises a plurality of stirring arms, each said stirring arm being mechanically connected, at a first end, to a stir junction and to one of said plurality of sensors at a second end.

12. The placement mechanism of claim 11, wherein each said second end is mechanically connected to a respective said sensor via a rotating joint that rotates around an axis perpendicular to a respective said stirring arm.

13. The placement mechanism of claim 11, wherein at least one of said plurality of stirring arms is telescopic.

14. A method for placing at least one sensor for non invasive measurement of at least one parameter, comprising:
providing at least one ultrasound transducer having a front side contact zone configured for being in contact with at least one sensor positioning site on the skin of a target patient;
attaching said at least one ultrasound transducer to a body of said target patient;
identifying signal based location indication by analyzing an ultrasound signal received from said ultrasound transducer;
adjusting, according to said signal based location indication, a signal capture angle of said at least one sensor in relation to said skin while maintaining said front side contact zone in contact with said target area; and
fixating said at least one sensor in said signal capture angle, while maintaining said front side contact zone in contact with said target area.

15. The method of claim 14, wherein said identifying comprises extracting a blood motion and a tissue motion from said ultrasound signal and performing said identifying by identifying a correlation between said blood motion and said tissue motion.

16. The method of claim 14, wherein said signal based location indication is a trace indicative of a mitral valve opening which is extracted from said ultrasound signal.

17. The method of claim 14, further comprising monitoring a plurality of cardiac parameters according to an analysis of the ultrasound signal.

18. The method of claim 14, further comprising identifying said at least one sensor positioning site according to a time velocity Doppler trace of an echocardiography imaging probe.

19. The method of claim 14, wherein said adjusting comprises presenting a plurality of indications each indicative of the detection of an interim signal based locator in said ultrasound signal.

20. The method of claim 14, further comprising acquiring an electrocardiogram (ECG) signal; wherein said adjusting is performed according to said ECG signal.

21. The method of claim 14, wherein said identifying comprises identifying the following:
a) systolic and diastoles in said ultrasound signal;
b) an indication of a mitral valve opening in said ultrasound Doppler signal during said diastole;
c) a plurality of onsets of a blood inflow in said ultrasound Doppler signal during said diastole; and
d) a repetitive pattern of said plurality of onsets during at least one respiration cycle of said target patient;
wherein each member of said a-d may not be identified if a proceeding member of said a-d has been identified.

22. A method of aiming at least one ultrasound transducer for measuring at least one parameter, comprising:
attaching at least one ultrasound transducer to a body of a target patient in front of at least one sensor positioning site;
receiving an ultrasound Doppler signal from said at least one ultrasound transducer;
tuning a transmission/reception angle of said at least one ultrasound transducer until the following are being identified:
a) systolic and diastoles in said ultrasound Doppler signal;
b) an indication of at least one mitral valve opening in said ultrasound Doppler signal during said diastole,
c) a plurality of onsets of a blood inflow in said ultrasound Doppler signal during said systole,
d) a repetitive pattern of said plurality of onsets during at least one respiration cycle of said target patient, and
fixating said at least one ultrasound transducer in said tuned transmission/reception angle in front of said at least one sensor positioning site;
wherein each member of said a-d may not be identified if a proceeding member of said a-d has been identified.

23. The method of claim 22, wherein said tuning is performed while maintaining a front side contact zone of each said ultrasound transducer in contact with a respective said transducer positioning site.

24. A method for monitoring a patient, comprising:
fixating each of a plurality of ultrasound transducers in a signal capture angle so that a front side contact zone thereof is in contact with one of a plurality of sensor positioning sites on the skin of a target patient; and
non invasively measuring, using said plurality of ultrasound transducers, at least one cardiac parameter in a plurality of sessions;
wherein each one of said plurality of ultrasound transducers remains fixated in a respective said signal capture angle during said monitoring period.
